(19) United States
(12) Patent Application Publication
(10) Pub. No.: US 2009/0299469 A1
(43) Pub. Date: Dec. 3, 2009

(54) CONE-SHAPED AORTIC ROOT REPLACEMENT GRAFT AND METHODS FOR MAKING AND USING SAME

(75) Inventor: Andras C. Kollar, Galveston, TX (US)

Correspondence Address: ROBERT W STROZIER, P.L.L.C, PO BOX 429, BELLAIRE, TX, 77402-0429, US

(73) Assignee: THE BOARD OF REGENTS OF THE UNIVERSITY OF TEXAS SYSTEM, Austin, TX (US)

(21) Appl. No.: 12/127,639

(22) Filed: May 27, 2008

(51) Int. Cl. A61F 2/06

(52) U.S. Cl. 623/2.12; 623/2.13; 623/2.14

(57) ABSTRACT

A new aortic root replacement graft or apparatus is disclosed and method for making and using same. The graft or apparatus includes a substantially straight and uniform cylindrical conduit having an outwardly flared end section so that a diameter of the cylindrical section is less than a diameter of a distal end of the flared end section.

### BACKGROUND OF THE INVENTION

1. Field of the Invention

The present invention relates to a new aortic root replacement graft or apparatus and method for making and using same.

More particularly, the present invention relates to a new aortic root replacement graft or apparatus and method for making and using same, where the graft or apparatus includes a substantially straight and uniform cylindrical conduit having an outwardly flared end section so that a diameter of the cylindrical section is less than a diameter of a distal end of the flared end section. The present invention also relates to method for using the aortic root replacement apparatus.

2. Description of the Related Art

The normal internal human aortic root conduit includes a sinus portion having three sinuses (bulges) surrounding the aortic valve, where the sinuses are called sinuses of Valsalva. The sinuses are arranged so that a cross-section of the sinus portion has a generally trefoil shape. The diameter and orifice area of the root are greater at the level of the sinus. The diameter and orifice area decrease slightly at the level below the valve called the basal ring (BR). The diameter and orifice areas decrease even more significantly (10% to 20% compared to BR) at the level ofthe sinotubular junction (STJ). The sinus portion connects to the ascending portion (above the level of the aortic valve) of the aorta which arborizes and distributes blood to the rest of the body. The heart's own blood vessels, called coronary arteries arise from the sinus portion (typically from two different sinuses).

The typical aortic valve has three “semilunar” leaflets, that open and close freely acting as a one way valve directing blood flow from the heart towards the body and preventing blood getting back to the heart when it is relaxed. These three leaflets are attached to the wall of the aortic root alongside a “coronet” shaped reinforced fibrous structure called the aortic “annulus”. This aortic annulus is a three dimensional structure—in comparison the above described BR and STJ are circular structures at the two ends of the aortic root conduit as shown in FIG. 1.²

The three leaflets, however, and similar to the corresponding sinuses and almost never fully equal in size. Most importantly, the height and width of these leaflets and the corresponding sinuses are typically different, therefore the BR and STJ rings are not parallel with each other creating further asymmetry along the aortic root's longitudinal axis.³⁻⁵

In addition, there is a common anatomical variation (in approximately 1-2% of all humans) where the aortic valve has only two leaflets (called bicuspid aortic valve). The significance of this variation is that this condition represents an impairment of the normal valve function and very often leads to valve malfunction with abnormal pressure dynamics and subsequent enlargement of the aortic root conduit (called aortic root aneurysm).⁶

The sinotubular junction (STJ) or sinus ridge and the sinuses of Valsalva are known to be crucial for the normal function of the aortic valve. The sinus ridge is important in causing initial fluid flow eddies inside the sinuses of Valsalva. During systole (the muscular contraction of the heart ejects blood from the main chamber of the heart towards the aorta), the aortic valve opens and eddy currents created prevent the leaflets of the aortic valve from impacting on the aortic wall. Then, during diastole (when the heart is relaxing), the eddy currents inside the sinuses cause the leaflets of the aortic valve to close so that blood cannot flow backwards; thus, acting as a one way valve mechanism. The sinus curvature is also important in sharing stress with the leaflet. It has been demonstrated that during systole, the sinus walls move outwardly (total sinus/commissural area expansion may exceed 50% in animal studies^{7,8}), and during diastole the sinus walls move inwardly taking up part of the load placed on the leaflet.

It is known that the longitudinal length of the sinuses changes very little during the cardiac cycle. In other words during the functioning of the aortic valve, the sinus sinuses move up and down as a whole without changing their length.⁷

For patients having aortic root aneurysm or dissection involving the aortic root and associated with aortic valve disease, the standard surgical approach has been to replace the aortic valve and ascending aorta by means of a composite vascular tube and valve graft onto which the two coronary artery orifices are reattached.^{9,10}

If the aortic valve leaflets are normal or only damaged to a small degree only (which is often the case with aneurysms) a so called valve-sparing aortic root reconstruction procedure that is designed to keep the patient own valve on site is a better alternative compared to using and artificial valve. The first such operation was designed by Magdi Yacoub in England,^{11,12} and it is called aortic root “remodeling” In this operation the enlarged sinus wall portions of the enlarged aortic root (right above the coronet shaped aortic annulus) are excised, then a straight tube graft (made of DACRON fibres) is tailored with three longitudinal tongues to replace the three sinuses FIGS. 2a & b. With the cylindrical graft the ultimate “bulging” effects are only moderate, but a more important point is that in this operation the pathologically weakened true aortic annulus is not supported and/or reconstructed and is likely to enlarge further in the long run.

In order to stabilize the three dimensional coronet shaped aortic annulus David and Feindel¹³ described a different surgical technique where the dilated aortic root is replaced with a straight tube graft (made of DACRON fibers) in a way, that the entire aortic root-annulus-valve structure is pulled inside the graft. This method is generally known as the “David Type reimplantation aortic valve sparing procedure.” The method provides excellent annulus stability, however, the lack of sinuses in a straight tube graft was found to negatively influence proper valve function, with the consequent risk of decreasing valve longevity.

Thus, the David re-implantation technique using a straight tube without a sinus component raises several problems, i.e., opening and closing of the native valve is not optimal. For example, upon valve opening, the leaflets might impact on the graft and be potentially damaged and the absence or delay in eddy current formation might alter valve closure as well and clearly increases the stress on the valve leaflets. The diastolic stress is borne only by the leaflet and is not shared with the sinuses causing a potential decrease in leaflet longevity.¹⁴

An optimal design for root replacement should therefore incorporate sinuses and a STJ, but at the same time it should allow the surgeon to adjust the conduit to the actual asymmetric root anatomy.

U.S. Pat. No. 5,139,515 disclosed an aortic graft having lower portions provided with “bulges” apparently mimicking the sinuses of Valsalva (Robicsek-Thubrikar graft—FIG. 3). However, no method to produce such a conduit for use in aortic surgery is described in the patent. U.S. Pat. No. 5,139,515 described a conduit having an “annular wall of a crimped material similar to that of conventional prosthesis”. No indication is actually given of how to obtain the “annularly-spaced radially outward bulges” mimicking the sinuses.

Moreover the drawings clearly show that the conduit, including the sinus portion, is provided along its whole length with corrugations which lie perpendicularly to the longitudinal axis of the prosethesis, and which impart longitudinal elasticity to the whole of the conduit.

The surgical technique described with this aortic root prosthesis is the exact same remodeling procedure as described by Yacoub (suturing the conduit above the coronet shaped aortic annulus), and therefore the conduit does not address the problems described with that operation. In addition the conduit is designed to be symmetric (with three equal “neo-sinuses”) and it may not be a good match for the typically asymmetric human aortic root. Moreover it cannot be used in the cases when a bicuspid valve can be spared during aneurysm surgery.

U.S. Pat. No. 6,352,554 disclosed a prosthetic aortic conduit including a first tubular portion and a second tubular portion connected together along a substantially common axis (De Paulis graft—FIG. 4). The second tubular portion does not substantially deform in a longitudinal direction and has resilient means which allow said second portion to be expandable in a lateral direction. As the second portion is able to deform laterally it is able to mimic the function of the sinuses of Valsalva. But again this conduit suffers from ineffective conformance with the typically asymmetric human aortic root and valves.

Thus, there is still a need for an effective prosthetic conduit to replace the aortic root while providing all the advantages of the natural sinuses of Valsalva.

### SUMMARY OF THE INVENTION

The present invention provides a prosthetic aortic apparatus that overcomes drawbacks mentioned above and is adapted to expand radially outwardly after implantation, while maintaining a degree of flexibility in the longitudinal direction, where the apparatus includes a cylindrical conduit having an outwardly flared end portion. The apparatus is specifically designed that after cutting the flared portion longitudinally and tailoring them into appropriate size “tongues” (based on precise measurements made during surgery) the conduit replaces the sinus portions matching the corresponding leaflets. Therefore, this conduit closely mimics the sinuses of Valsalva, and their action during a cardiac cycle and to permit the formation of bulges during implantation. In addition, there is a new suture technique described in details by the inventor recently¹⁵ that incorporates a stabilizing method for the three dimensional aortic annulus (in essence the annulus is sandwiched alongside the coronet in between a layer of graft material and “pledgets”).

In certain embodiments, the flared end portion comprises a constant angle flare. In other embodiments, the flared end portion comprises a compound flare have a plurality of flared sections having different flare angles. In other embodiments, the flared end portion comprises a complex compound flare having sections having constant flare angle and section having varying flare angles.

The present invention also provides a new aortic root replacement graft or apparatus including a substantially straight cylindrical conduit having an outwardly flared end section so that a diameter of the cylindrical section is less than a diameter of a distal end of the flared end section.

The present invention also provides a prosthetic aortic apparatus for replacing a root portion of an aorta, where the apparatus includes a substantially straight and uniform cylindrical conduit and an outwardly flared conduit disposed at a distal end of the cylindrical section, where a small end of the flared conduit is affixed to an end of the cylindrical conduit. The apparatus is characterized in that a diameter of the cylindrical section (d₁) is less than a diameter of a distal end of the flared conduit (d₂). The flared conduit can also be adapted to insubstantially deform in its longitudinal direction or to deform in its longitudinal direction to a desired extent. The flared conduit can also be laterally resilient allowing it to expand in its lateral direction. This lateral deformability of the flared conduit permits it to mimic the function of the sinuses of Valsalva, while the conical shape of the flared conduit permits the formation of bulges during implantation to support eddy currents keeping the valve leaflets from impacting the wall of the conduit. In certain embodiments, the flared conduit is conically shaped so that the flare is uniform, i.e., the flare extends from the cylindrical conduit at a constant flare angle.

The present invention also provides a prosthetic aortic conduit for replacing a root portion of an aorta including a substantially straight and uniform cylindrical section and a conically shaped end section, where a small end of the end section is affixed to an end of the cylindrical section. The apparatus is characterized in that a diameter d₁ of the cylindrical section is less than a diameter d₂ of a distal end of the cone-shaped end section. The conically shaped end section can also be adapted to insubstantially deform in its longitudinal direction or to deform in its longitudinal direction to a desired extent. The conically shaped end section can also be laterally resilient allowing it to expand in its lateral direction. This lateral deformability of the conical shaped end section permits it to mimic the function of the sinuses of Valsalva, while the shape of the conically shaped end section permits the formation of bulges during implantation to support eddy currents keeping the valve leaflets from impacting the wall of the sections.

The present invention also provides a method of manufacturing a prosthetic aortic conduit including the step of providing a substantially uniform cylindrical conduit section suitable for use in heart surgery, the cylindrical conduit section has a longitudinal axis and optionally is resilient or has a resilient means allowing some expansion in its longitudinal direction. The method also includes the step of securing to an end of the cylindrical conduit section, an outwardly flared conduit section also suitable for use in heart surgery so that the cylindrical section and the flared section align and smoothly transition from one to the other. The flared conduit section also has a longitudinal axis and optionally is resilient or has a resilient means allowing the flared section to expand in its lateral direction.

### BRIEF DESCRIPTION OF THE DRAWINGS

The invention can be better understood with reference to the following detailed description together with the appended illustrative drawings in which like elements are numbered the same:

FIG. 1 depicts a expanded view of the three dimensional geometrical arrangement of the anatomical components of the aortic root.

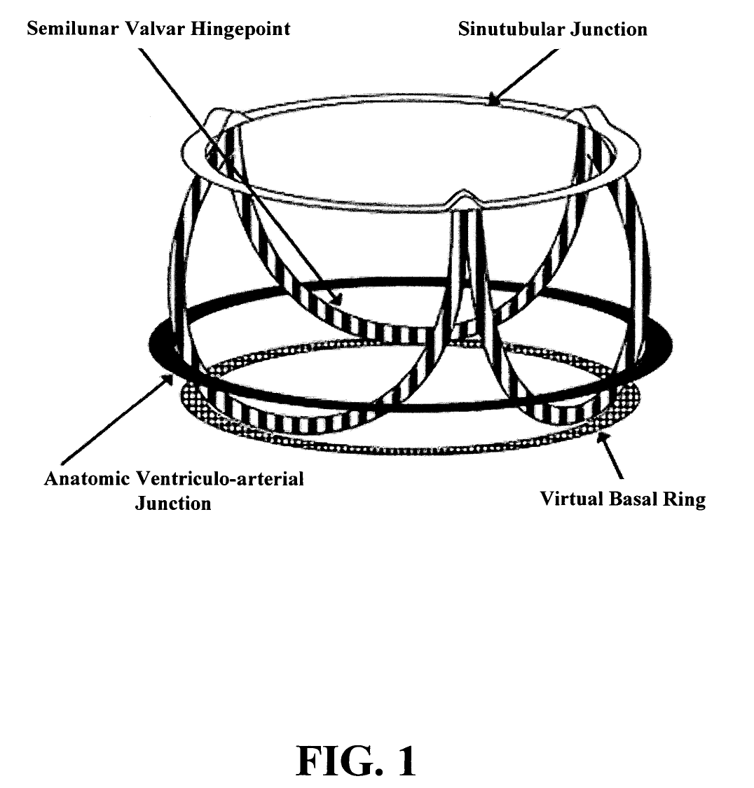

FIGS. 2A & B depict the prior art of the so called “remodeling” technique designed by Magdi Yacoub utilizing a straight tube graft to replace the three aortic sinuses (the graft is sutured to the “residual sinus wall above the true coronet shape annulus).

FIG. 3 depicts the prior art Robicsek-Thubrikar graft.

FIG. 4 depicts the prior art De Paulis graft.

FIG. 5 depicts an embodiment of a prosthetic aortic conduit of this invention.

FIG. 6 depicts another embodiment of a prosthetic aortic conduit of this invention.

FIG. 7 depicts another embodiment of a prosthetic aortic conduit of this invention.

FIG. 8 depicts another embodiment of a prosthetic aortic conduit of this invention.

FIGS. 9A & B depict another embodiment of a prosthetic aortic conduit of this invention.

FIGS. 10A & B depict another embodiment of a prosthetic aortic conduit of this invention.

FIGS. 11A & B depict another embodiment of a prosthetic aortic conduit of this invention.

FIGS. 12A-G depict a series of cuts that can be made in the conical section of the prosthetic aortic conduit of this invention to accommodate hearts having a non-limiting relative cusp area classification.

### DETAILED DESCRIPTION OF THE INVENTION

The inventor has found that a new prosthetic aortic apparatus can be constructed, where the apparatus includes at least two major sections a substantially straight cylindrical section and an outwardly flared end section or conical end section. The sections can be adapted to have elasticity in the lateral and/or longitudinal directions as desired. The elasticity can either be an aspect or characteristic of the material out of which the sections are constructed or the sections can be constructed so that their structure supports elasticity in one or more directions. The apparatus can be a unitary construction having different materials or material orientations in the different sections or the apparatus can be a construction were two or more sections are affixed together to form the final apparatus. The apparatuses can also include an artificial valve associated with the flared section if valve replacement is also warranted; of course, the natural aortic valve/annulus complex can be attached to the apparatus during surgical implantation.

The present invention broadly relates to a prosthetic aortic apparatus including a substantially straight cylindrical conduit having an outwardly flared end section, where a diameter d₁ of the cylindrical section is less than a diameter d₂ of a distal end of the flared end section and where the two section are affixed to each other at a proximal end of the flared section.

In certain embodiments, a length (l) of the flared or conical end section is less than or equal to d₂. In certain embodiments, d₂ ranges from about 1.1×d₁ to about 1.5×d₁, regardless of the relationship between l and d₂. In other embodiments, d₂ ranges from about 1.1×d₁ to about 1.4×d₁, regardless of the relationship between l and d₂. In other embodiments, d₁ ranges from about 1.1 ×d₁ to about 1.3×d₁, regardless of the relationship between l and d₂. In other embodiments, d₂ ranges from about 1.15×d₁ to about 1.3×d₁, regardless of the relationship between l and d₂.

In certain embodiments, the resilient means associated with the flared end section includes longitudinally extending corrugations. In other embodiments, the resilient means associated with the cylindrical conduit of the invention comprises circular corrugations successively provided along the longitudinal axis of the conduit. In other embodiments, the cylindrical conduit and the flared end portion comprise two distinct constructs secured together at one end of cylindrical conduit and the small diameter end of the flared end portion. In other embodiments, the apparatus can include a third portion connected to the large end of the flared portion, where the third portion is either flared or substantially straight. In certain embodiments, the third portion is provided with resilient means which allows expansion of the third portion in a longitudinal direction. In other embodiments, the apparatuses of this invention can include a prosthetic valve. In other embodiments, the resilient means associated with the cylindrical section includes a plurality of annular corrugations successively provided along the longitudinal axis of the conduit and the resilient means associated with the flared section includes a plurality of longitudinally extending corrugations successively provided around the circumference of the flared end portion.

The present invention broadly relates to method for making an apparatus of this invention, including the step of providing a substantially uniform cylindrical conduit section suitable for use in heart surgery, the cylindrical conduit section has a longitudinal axis and optionally is resilient or has a resilient means allowing some expansion in its longitudinal direction. The method also includes the step of securing to an end of the cylindrical conduit section, an outwardly flared conduit section also suitable for use in heart surgery so that the cylindrical section and the flared section align and smoothly transition from one to the other. The flared conduit section also has a longitudinal axis and optionally is resilient or has a resilient means allowing the flared section to expand in its lateral direction. Alternatively, the apparatus can be constructed as a unitary construct where the material is formed in the desired shape with resiliency residing either in the inherent nature of the material or in corrugations (longitudinally or laterally) or other means for adding longitudinal or lateral resiliency.

Where a third tubular conduit is required, the third conduit section is attached to the end of the flared conduit section. One end of the third conduit should align with the distal end of the flared conduit so that third conduit can be affixed to the flared conduit. The third conduit may also be attached to a combined cylindrical and flared construct or the third conduit may be attached to the flared conduit prior to attaching the cylindrical conduit to the flared conduit at its other end. In certain embodiments, the third conduit will have circumferentially extending corrugations for longitudinal resiliency. In most embodiments, the third conduit will have length less than the length of the flared conduit.

Suitable materials for construction of the apparatus of this invention include, without limitation, a non-erodible bio-compatible polymer approved for aortic replacement surgery. Exemplary examples include, without limitation, a polyester material, an expandable polyester material, a polytetrafluoroethylene (PTFE) material, an expanded PTFE material, or other similar polymers. In certain embodiments, the apparatuses are constructed from a fabric like DACRON or other similar fabrics. In other embodiments, the conduits of the apparatuses are made of DACRON and/or a PTFE material.

### Context of the Invention

The aortic root is a complex anatomical structure. Starting at the level of the ventriculo-aortic junction with a muscular fibrous basal “ring” (BR) part of the structure, the root terminates just above the valve commisures in a well defined fibrous “ridge” called sinotubular junction (STJ). Typically, there are three semilunar shaped aortic valve leaflets suspended between these two levels alongside a “coronet” shaped fibrous ridge called the aortic “annulus”. The other unique feature of the aortic root is the significant “outpouching” of its free walls called the “Valsalva sinuses”. Corresponding to the three leaflets, there are three sinuses essentially mirroring the semilunar valve leaflets, and they are believed to play important roles in valve closure (eddy currents), leaflets stress sharing and also in the in the maintenance of coronary blood flow.

The simplified geometry of the normal aortic root is of a truncated cone (the diameter of the STJ is 10% to 20% smaller than the BR) and this geometrical arrangement is believed to play a fundamental role in maintaining valvular competence.¹ The cross section of the aortic root, however, changes from circular at the BR to “clover shape” at the Valsalva sinuses and back to circular again at the STJ and mimicking this unique architecture in root replacement surgery has been extremely difficult.

Another challenge is that the three semilunar valve leaflets and the corresponding sinuses are almost never equal.³⁻⁵ In particular, the height and width of the leaflets/sinuses are typically different which creates additional asymmetry along the long axis of the normal aortic root. Therefore, pre-manufacturing replacement grafts to match these individual variations has not been possible.

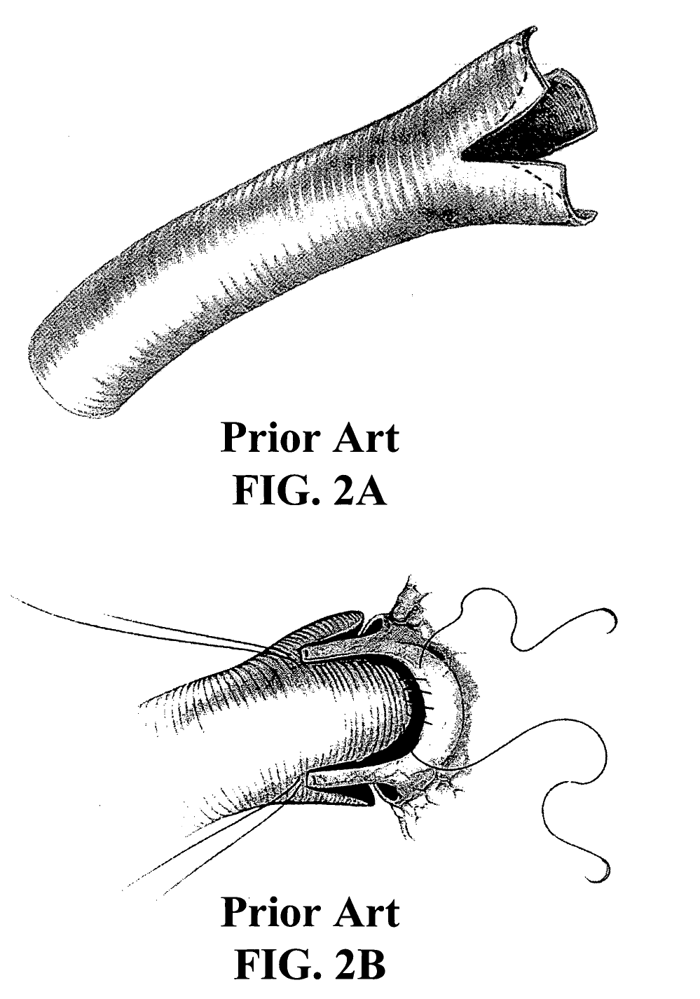

Historically, aortic root replacement was performed with a straight tube graft containing an artificial valve.^{9,10} Today, the state of the art surgical techniques for patients with aortic root aneurysm are the various “valve sparing” operations, that were originally utilizing a straight tube graft (in the 80^{th} and early 90^{th} no other graft was available). In the so called “remodeling” procedure designed by Yacoub^{11,12} the graft is tailored with three “tongues” to replace the Valsalva sinuses as shown in FIGS. 2A & B, but the bulging effects are moderate. Another problem with this technique is that the replacement “tongues” are sutured to the residual sinus wall and not directly to the fibrous aortic annulus imparting no stabilizing effect on the already weakened structure.

In the “re-implantation” technique invented by David,¹³ the entire aortico-ventricular junction and valve suspension apparatus are pulled inside an intact (non-tailored) straight tube graft. This maneuver unequivocally stabilizes the coronet shaped annulus, but there is complete loss of the out-pouching sinus structures. Ever since the introduction of these two techniques there has been a search for surgical modifications and for new graft designs that mimic the original triple bulge pattern of the normal aortic root.

More recently, two patented aortic root grafts were introduced into clinical practice—one for the remodeling operation and another for reimplantation. Both were designed to facilitate larger neo-sinus creation, but follow a different concept.

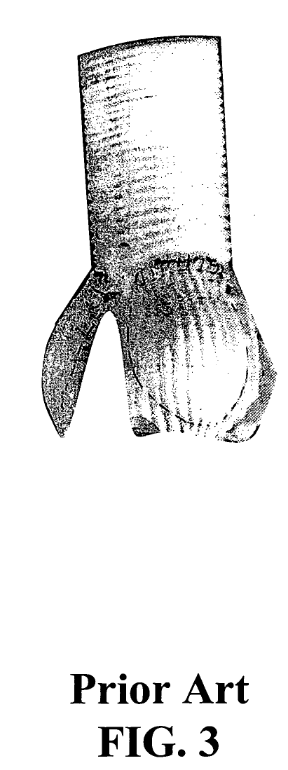

The “Robicsek-Thubrikar” graft¹⁶ has three equal size teardrop shaped extensions attached to the bottom of the straight tube graft and the excess material creates larger size neo-sinuses as shown in FIG. 3. This graft, however, is not a good match for the typically asymmetric root structures and it could not be used at all in cases of bicuspid aortic valves.

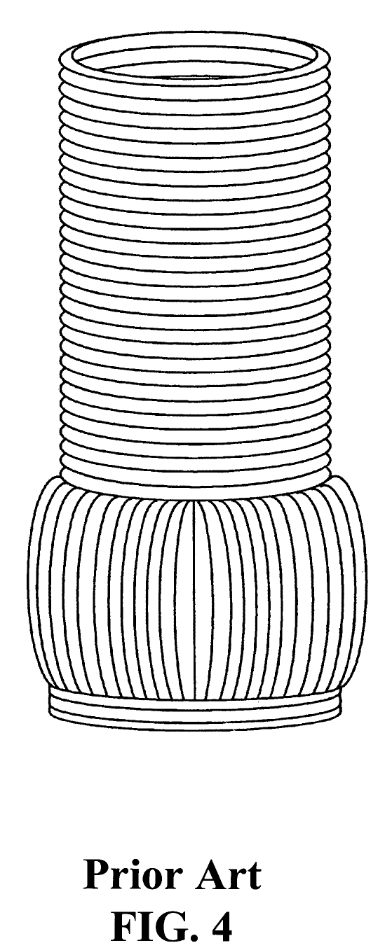

The “De Paulis” graft¹⁷ designed for re-implantation has a circumferentially ballooning segment called “skirt” incorporated at the base of the graft as shown in FIG. 4, and the aortic valve commissures are to be re-suspended within this segment imitating neo-sinuses. The design does not follow the triple bulge concept and due to this and possibly as a result of the distortion of the normal “cone” geometry, this operation can be technically difficult.

From personal experience with a new operative procedure that integrates the surgical principles of the above described two aortic root replacement techniques,^{15,18} I propose a different shape aortic root graft. My concept is that graft design should be fairly simple to allow easy manufacturing in different size and at the same time, after individual tailoring, it should match the patient's actual anatomy with excess graft material left to create bulging neo-sinuses.

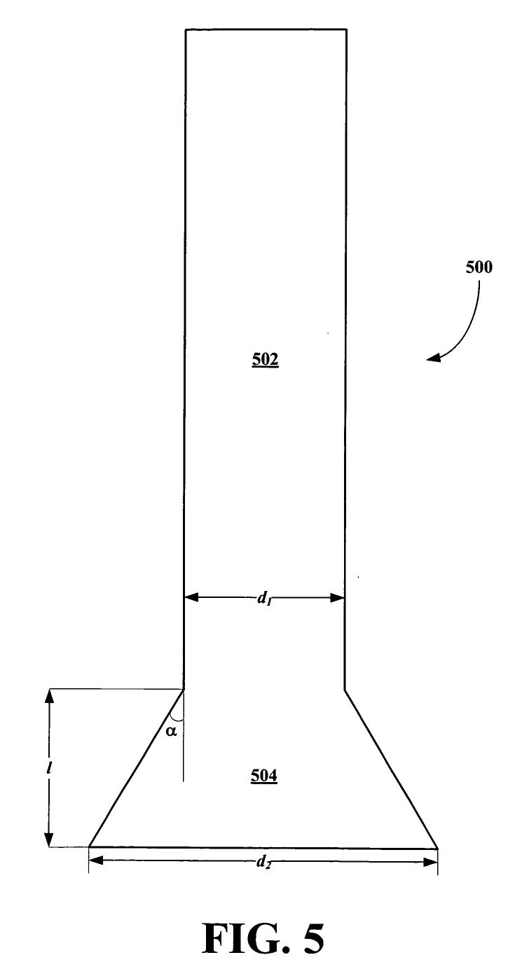

My invention comprises straight tube graft including a truncated cone shaped end, one embodiment of the graft is shown in FIG. 5. The diameter of the tube represents the restored STJ and the excess graft material of the widening “cone” can be individually tailored by the surgeon (using the remodeling method) to create (a) different size “tongues” if necessary to match the actual anatomy and (b) larger size neo-sinuses—bulges. In essence the new graft design follows the geometrical pattern (cone shape) of the normal human aortic root.

In addition, I have recently described a new suture technique in details¹⁵ that incorporates a stabilizing methodology for the three dimensional coronet shaped aortic annulus (without pulling the structure inside a rigid tube to provide external stabilization). According to my technique the annulus is sandwiched in between the tailored graft material on one side and “pledgets” on the other side and this suture line follows the coronet.

This new graft design could be used for aortic root replacement in cases of aneurysm, but if the manufacturing problems of durable elastic materials are solved, then such a graft will be useful in pulmonary root autograft (Ross procedure). The pulmonary root dilates when exposed to the higher blood pressure conditions in the aorta and by “wrapping it externally with an elastic “cone shape” graft would prevent dilatation of this conduit in adults.

### DETAILED DESCRIPTION OF EMBODIMENTS OF THE INVENTION

Referring now to FIG. 5, an embodiment of an aortic conduit apparatus of this invention, generally 500, is shown to include a substantially straight cylindrical conduit section 502 having a flared end section 504. The sections can each be made of DACRON, but any other suitable biocompatible material such as polytetrafluoroethylene (PTFE) could be used as well. Additionally, the two sections can be made of different materials. The cylindrical section 502 have a diameter of d₁. The flared end section 504 has a length l, a proximal diameter substantially the same as the diameter d₁ of the cylindrical section 502, and a distal end diameter d₂. The apparatus 500 is characterized in that d₁ is less than d₂ and l is the same or equal to d₂ or l is greater than d₁, but shorter than d₂. In this embodiment, the apparatus 500 is a unitary construct, where the cylindrical section transitions smoothly and seamlessly into the flared section. In certain embodiments, the two section are made of the same material, while in other embodiments, the material may be different. In other embodiments, the two section can comprise material with orientable fibers, the cylindrical section having fibers oriented to permit longitudinal elasticity and in the flared section fibers oriented to permit lateral elasticity. Alternatively, the flare can be defined with respect to an angle, α, the flare make with an longitudinal edge of the cylindrical section. The angle α ranges from about 5° to about 45°. In certain embodiments, the angle α ranges from about 10° to about 30°. In other embodiments, the angle α ranges from about 15° to about 30°.

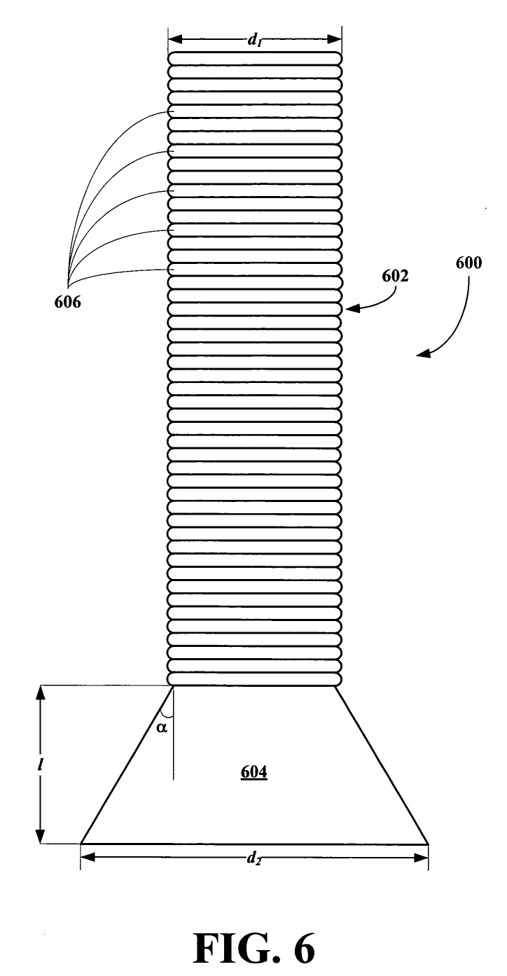

Referring now to FIG. 6, an embodiment of an aortic conduit apparatus of this invention, generally 600, is shown to include a substantially straight cylindrical conduit section 602 and a flared end section 604. The sections can each be made of DACRON, but any other suitable biocompatible material such as polytetrafluoroethylene (PTFE) could be used as well. Additionally, the two sections can be made of different materials. The cylindrical section 602 have a diameter of d₁. The flared end section 604 has a length l, a proximal diameter substantially the same as the diameter d₁ of the cylindrical section 602, and a distal end diameter d₂. In this embodiment, the cylindrical section 602 is corrugated with corrugations 606, which permit longitudinal elasticity and comprise a resilient means to permit longitudinal elongation and restoration. Alternatively, the flare can be defined with respect to an angle, α, the flare make with an longitudinal edge of the cylindrical section. The angle α ranges from about 5° to about 45°. In certain embodiments, the angle α ranges from about 10° to about 30°. In other embodiments, the angle α ranges from about 15° to about 30°.

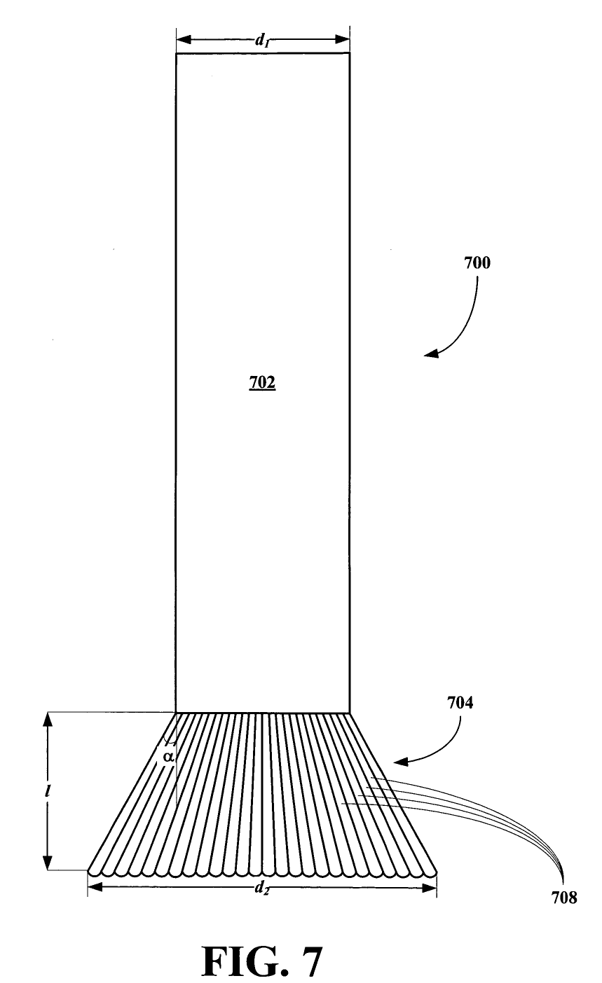

Referring now to FIG. 7, an embodiment of an aortic conduit apparatus of this invention, generally 700, is shown to include a substantially straight cylindrical conduit section 702 and a flared end section 704. The sections can each be made of DACRON, but any other suitable biocompatible material such as polytetrafluoroethylene (PTFE) could be used as well. Additionally, the two sections can be made of different materials. The cylindrical section 702 have a diameter of d₁. The flared end section 704 has a length l, a proximal diameter substantially the same as the diameter d₁ of the cylindrical section 702, and a distal end diameter d₂. Alternatively, the flare can be defined with respect to an angle, α, the flare make with an longitudinal edge of the cylindrical section. The angle α ranges from about 5° to about 45°. In certain embodiments, the angle α ranges from about 10° to about 30°. In other embodiments, the angle α ranges from about 15° to about 30°. In this embodiment, the flared section 704 is corrugated with corrugations 708, which permit lateral elasticity and comprise a resilient means to permit lateral elongation and restoration.

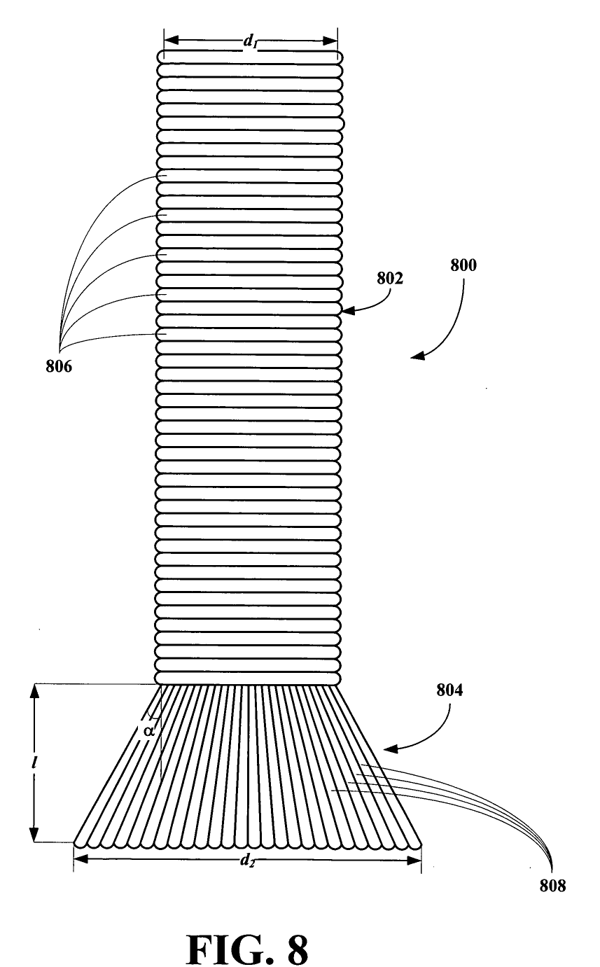

Referring now to FIG. 8, an embodiment of an aortic conduit apparatus of this invention, generally 800, is shown to include a substantially straight cylindrical conduit section 802 and a flared end section 804. The sections can each be made of DACRON, but any other suitable biocompatible material such as polytetrafluoroethylene (PTFE) could be used as well. Additionally, the two sections can be made of different materials. The cylindrical section 802 have a diameter of d₁. The flared end section 804 has a length l, a proximal diameter substantially the same as the diameter d₁ of the cylindrical section 802, and a distal end diameter d₂. Alternatively, the flare can be defined with respect to an angle, α, the flare make with an longitudinal edge of the cylindrical section. The angle α ranges from about 5° to about 45°. In certain embodiments, the angle α ranges from about 10° to about 30°. In other embodiments, the angle α ranges from about 15° to about 30°. In this embodiment, the cylindrical section 802 is corrugated with corrugations 806, which permit longitudinal elasticity and comprise a resilient means to permit longitudinal elongation and restoration, and the flared section 804 is corrugated with corrugations 808, which permit lateral elasticity and comprise a resilient means to permit lateral elongation and restoration.

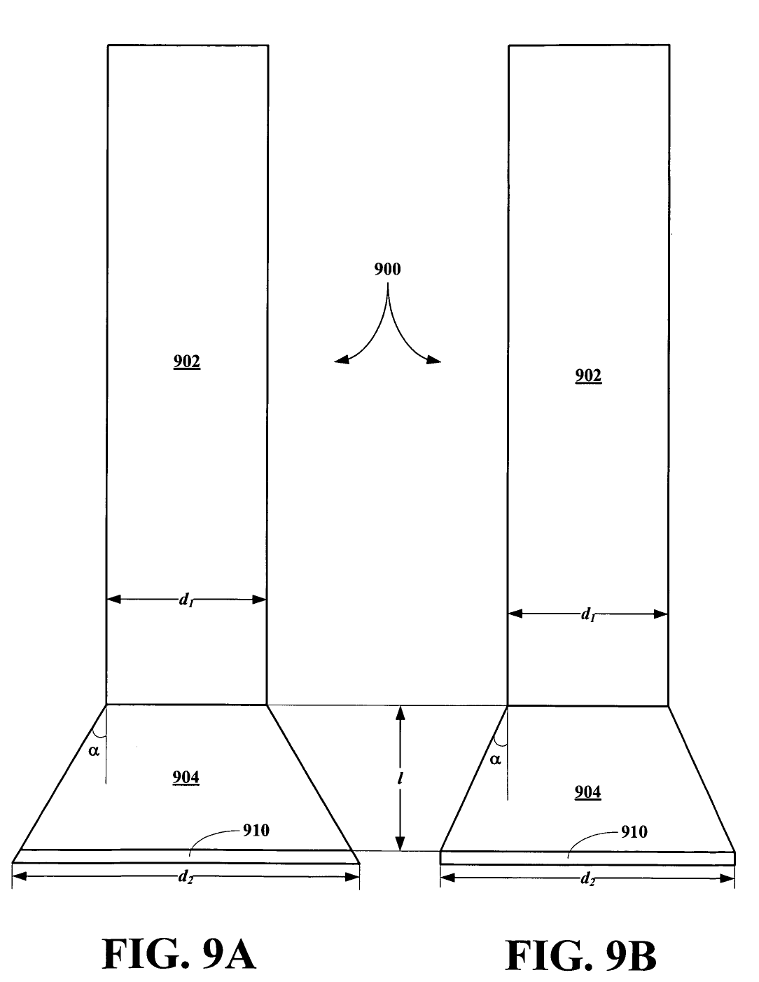

Referring now to FIGS. 9A & B, an embodiment of an aortic conduit apparatus of this invention, generally 900, is shown to include a substantially straight cylindrical conduit section 902, a flared end section 904 and a skirt section 910. The sections can each be made of DACRON, but any other suitable biocompatible material such as polytetrafluoroethylene (PTFE) could be used as well. Additionally, the two sections can be made of different materials. The cylindrical section 902 have a diameter of d₁. The flared end section 904 has a length l, a proximal diameter substantially the same as the diameter d₁ of the cylindrical section 902, and a distal end diameter d₂. Alternatively, the flare can be defined with respect to an angle, α, the flare make with an longitudinal edge of the cylindrical section. The angle α ranges from about 5° to about 45°. In certain embodiments, the angle α ranges from about 10° to about 30°. In other embodiments, the angle α ranges from about 15° to about 30°. In FIG. 9A, the skirt 910 is angled as the flared section 904 is angled. Although the angle of the skirt 910 is shown to be the same, it does not need to be. In fact, in FIG. 9B, the skirt 910 is rectangular shaped. The skirt 910 can be made of a different material than the flared section 804 for anchoring the apparatus 800 to the BR of the root.

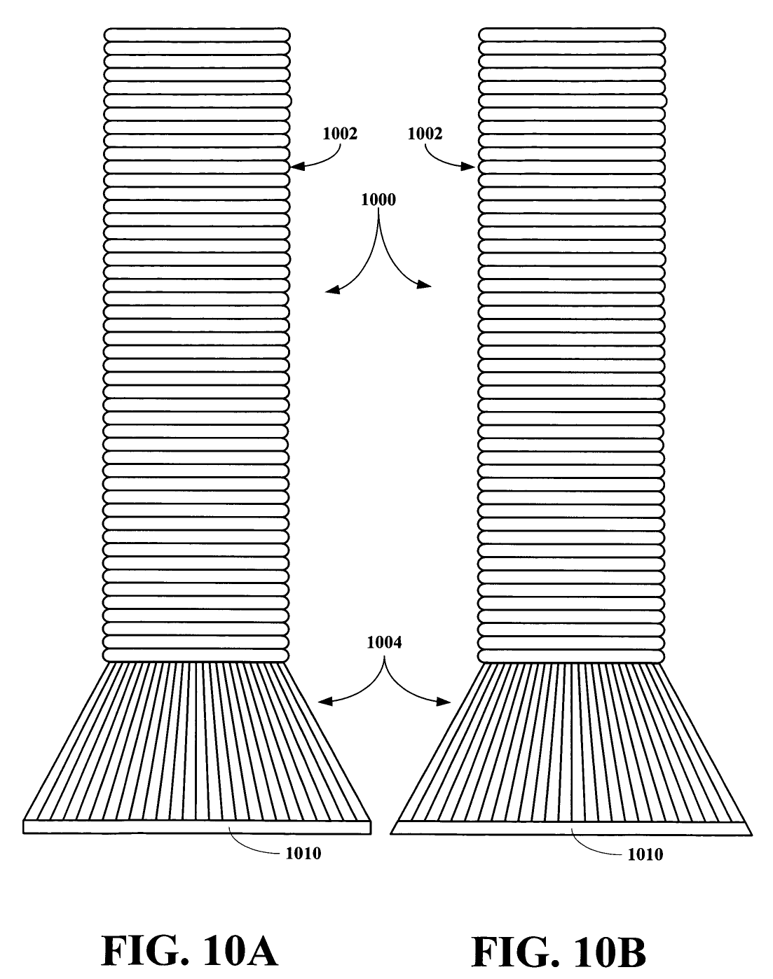

Referring now to FIGS. 10A & B, an embodiment of an aortic conduit apparatus of this invention, generally 1000, is shown to include a substantially straight and longitudinally corrugated cylindrical conduit section 1002, a flared and laterally corrugated end section 1004 and a skirt section 1010, where the longitudinal corrugated cylindrical section 1002 comprises a longitudinal resilient means and the lateral corrugated flared section 1004 comprises a lateral resilient means. The sections can each be made of DACRON, but any other suitable biocompatible material such as polytetrafluoroethylene (PTFE) could be used as well. In these embodiments, the apparatuses are characterized by same length, diameter and angle conditions set forth previously. In FIG. 10A, the skirt 1010 is angled as the flared section 1004 is angled. Although the angle of the skirt 1010 is shown to be the same, it does not need to be. In fact, in FIG. 10B, the skirt 1010 is rectangular shaped. The skirt 1010 can be made of a different material than the flared section 1004 for anchoring the apparatus 1000 to the BR of the root.

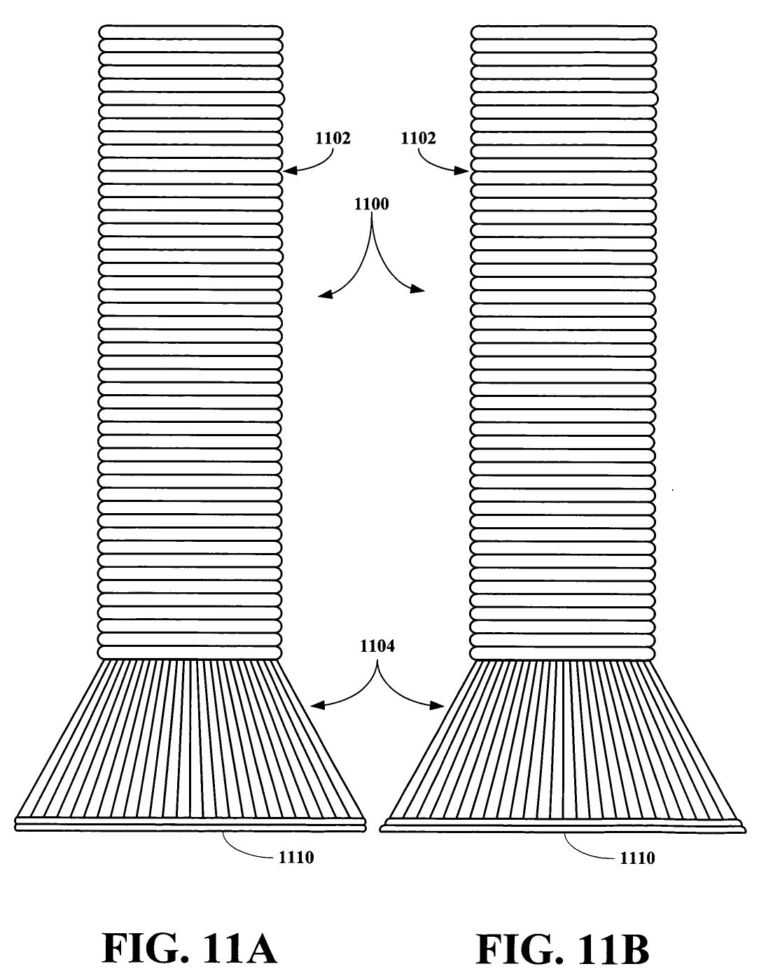

Referring now to FIGS. 11A & B, an embodiment of an aortic conduit apparatus of this invention, generally 1100, is shown to include a substantially straight and longitudinally corrugated cylindrical conduit section 1102, a flared and laterally corrugated end section 1104 and a longitudinally corrugated skirt section 1110, where the longitudinal corrugated cylindrical section 1102 comprises a longitudinal resilient means, the lateral corrugated flared section 1104 comprises a lateral resilient means and the longitudinal corrugated skirt 1110 comprises a second longitudinal resilient means. The sections can each be made of DACRON, but any other suitable biocompatible material such as polytetrafluoroethylene (PTFE) could be used as well. Additionally, the two sections can be made of different materials. In these embodiments, the apparatuses are characterized by same length, diameter and angle conditions set forth previously. In FIG. 11A, the skirt 1110 is angled as the flared section 1104 is angled. Although the angle of the skirt 1110 is shown to be the same, it does not need to be. In fact, in FIG. 11B, the skirt 1110 is rectangular shaped. The skirt 1110 can be made of a different material than the flared section 1104 for anchoring the apparatus 1100 to the BR of the root.

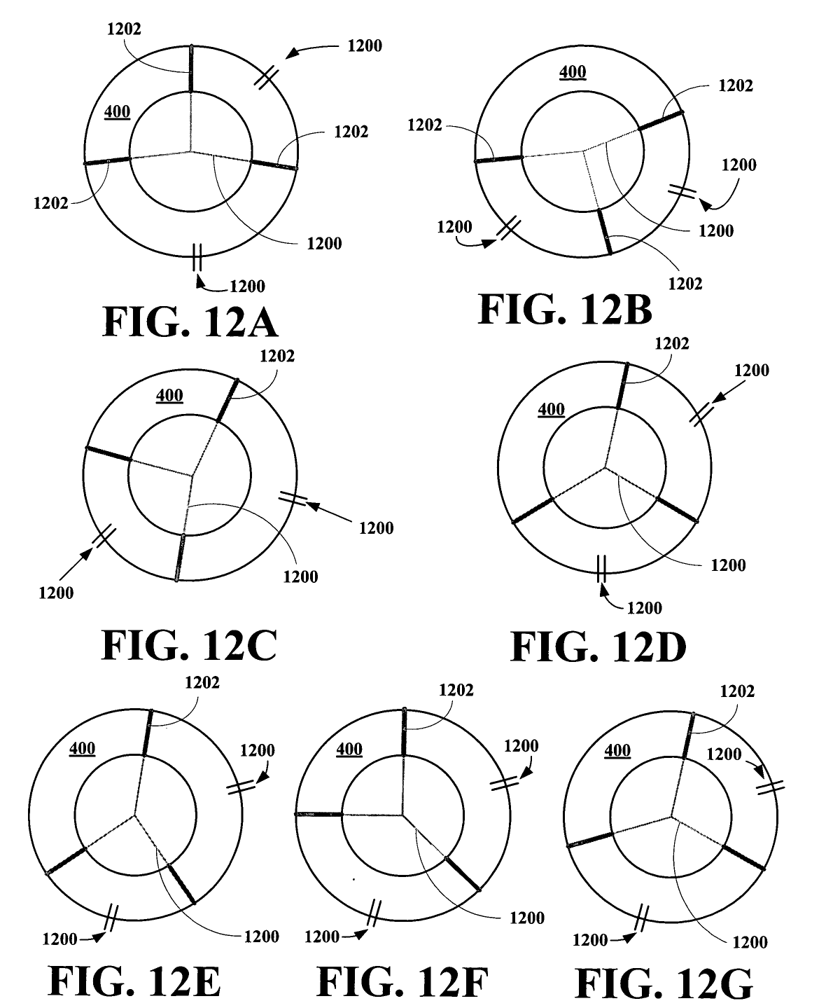

Referring now to FIGS. 12A-G, the apparatuses of any of the FIGS. 5-11 are shown here with heart cusp area classifications 1200 superimposed on the apparatus of FIG. 5 for illustration. Along with the classification lines, cut lines 1202 are shown for cutting the apparatuses of this invention for forming a conforming implant for root conduit replacement depending on the heart structure. Thus, the flared section of the apparatuses of this invention can be cut by the surgeon to conform to the structure of the patient's natural aortic root. These cuts permit the surgeon to fashion the replacement conduit so that the replacement will have bulges that conform to the patient's heart valve leaflets mimicking the replaced sinus Valsalva structures to a greater degree.

### REFERENCES CITED IN THE INVENTION

The following references were cited in the specification:
1. Reid K: The anatomy of the sinus of Valsalva. Thorax. 1970; 25:79-85.
2. Thubrikar M: The aortic valve CRC Press Inc, Boca Raton, Fla., 1990.
3. Vollebergh F E, Becker A E: Minor congenital variations of cusp size in tricuspid aortic valves. Possible link with isolated aortic stenosis. Br Heart J. 1977; 39:1006-11.
4. Choo S J, McRae G, Olomon J P et al: Aortic root geometry: pattern of differences between leaflets and sinuses of Valsalva. J Heart Valve Dis. 1999;8:407-15.
5. Berdajs D, Lajos P, Turina M: The anatomy of the aortic root. Cardiovasc Surg. 2002;10:320-7.
6. Braverman A C, Guven H, Beardslee M A, Makan M. Kates A M, Moon M R: The bicuspid aortic valve. Curr. Probl. Cardiol. 2005;30:470-522.
7. Swanson W M, Clark R E: Dimensions and geometric relationships of the human aortic valve as a function of pressure. Circ. Res. 1974; 55:871-82.
8. Lansac E, Lim H S, Shomura Y et al.: A four dimensional study of the aortic root dynamics. Eur. J. Cardio-Thor. Surg. 2002; 2:497-503.
9. Bentall H, De Bono A: A technique for complete replacement of the ascending aorta. Thorax. 1968; 23:338-9.
10. Cabrol C, Pavie A, Mesnildrey P et al.: Long-term results with total replacement of the ascending aorta and reimplantation of the coronary arteries. J Thorac Cardiovasc Surg. 1986;91:17-25.
11. Yacoub M H, Fagan A, Stassano P, Radley-Smith R: Results of valve conserving operations for aortic regurgitation (abstract). Circulation 1983; 68:311-2.
12. Sarsam M A, Yacoub M: Remodeling of the aortic valve annulus. J Thorac Cardiovasc Surg. 1993; 105:435-8.
13. David T E, Feindel C M: An aortic valve-sparing operation for patients with aortic incompetence and aneurysm of the ascending aorta. J Thorac Cardiovasc Surg. 1992;103:617-21.
14. Beck A, Thubrikar M J, Robicsek F: Stress analysis of the aortic valve with and without the sinuses of valsalva. J Heart Valve Dis. 2001; 10:1-11.
15. Kollar A: Valve-sparing reconstruction within the native aortic root: integrating the Yacoub and the David methods. Ann Thorac Surg. 2007; 83:2241-3.
16. Thubrikar M J, Robicsek F, Gong G G, Fowler B L: A new aortic root prosthesis with compliant sinuses for valve-sparing operations. Ann. Thorne. Surg. 2001;71:S318-22.
17. De Paulis R, De Matteis G M, Nardi P et al: One year appraisal of new aortic root conduit with sinuses of Valsalva. J. Thor. Cardiovasc. Surg. 2002;122:33-9.
18. Kollar A C, Lick S D, Conti V R: Integrating resuspension with remodeling: early results with a new valve sparing aortic root reconstruction technique. J Heart Valve Dis. 2008;17:74-80

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

I claim:

1. A prosthetic aortic apparatus comprising:
a cylindrical conduit including:
a circular first end,
a substantially straight section, and
an outwardly flared second end,
where the flared end is designed to be cut to closely mimic Valsalva sinuses of an aortic root of a patient to permit formation of bulges that expand radially outwardly and maintain a degree of flexibility in the longitudinal direction after implantation of the apparatus.

2. The apparatus of claim 1, wherein the flared end comprises a constant flare angle α ranging from about 5° to about 45°.

3. The apparatus of claim 2, wherein the angle α ranges from about 10° to about 30°.

4. The apparatus of claim 2, wherein the angle α ranges from about 15° to about 30°.

5. The apparatus of claim 1, wherein the flared end comprises a compound flare have a plurality of flared sections having different flare angles.

6. The apparatus of claim 1, wherein the flared end comprises a complex compound flare having sections having constant flare angle and section having varying flare angles.

7. A new aortic root replacement apparatus comprising:
a cylindrical conduit including:
a circular first end having a diameter d₁,
a substantially straight section having the diameter d₁, and
an outwardly flared second end having a length l and a diameter d₁,
where the flared end is designed to be cut to closely mimic Valsalva sinuses of an aortic root of a patient to permit formation of bulges that expand radially outwardly and maintain a degree of flexibility in the longitudinal direction after implantation of the apparatus.

8. The apparatus of claim 7, wherein the length l is less than or equal to the diameter d₂.

9. The apparatus of claim 7, wherein the diameter d₂ ranges from about 1.1×d₁ to about 1.5×d₁, regardless of the relationship between l and d₂.

10. The apparatus of claim 7, wherein the diameter d₂ ranges from about 1.1×d₁ to about 1.4×d₁, regardless of the relationship between l and d₂.

11. The apparatus of claim 7, wherein the d₂ ranges from about 1.1×d₁ to about 1.3×d₁, regardless of the relationship between l and d₂.

12. The apparatus of claim 7, wherein the d₂ ranges from about 1.15×d₁ to about 1.3×d₁, regardless of the relationship between l and d₂.

13. The apparatus of claim 7, wherein the length l is less than or substantially equal to the diameter d₂.

14. The apparatus of claim 7, wherein the diameter d₁ is less than the diameter d₂ and the length l is the less than or equal to the diameter d₂.

15. The apparatus of claim 7, wherein the length l is greater than the diameter d₁, but shorter than the diameter d₂.

16. The apparatus of claim 1, wherein the flared end comprises a constant flare angle α ranging from about 5° to about 45°.

17. The apparatus of claim 1, wherein the flared end comprises a compound flare have a plurality of flared sections having different flare angles.

18. The apparatus of claim 1, wherein the flared end comprises a complex compound flare having sections having constant flare angle and section having varying flare angles.

19. A prosthetic aortic apparatus for replacing a root portion of an aorta, comprising:
a tubular conduit including:
a substantially straight and uniform section having a diameter d₁, and
an outwardly flared section disposed at a distal end of the straight section having a length l and a diameter d₂ at the open flared end,
where a distal end of the flared section is designed to be cut to closely mimic Valsalva sinuses of an aortic root of a patient to permit formation of bulges that expand radially outwardly and maintain a degree of flexibility in the longitudinal direction after implantation of the apparatus.

20. The apparatus of claim 19, wherein the flared section is affixed to an end of the straight section.

21. The apparatus of claim 19, wherein the flared conduit section is adapted to insubstantially deform in its longitudinal direction and is adapted to deform in its lateral direction.

22. The apparatus of claim 19, wherein the flared conduit section is adapted to deform in its longitudinal direction to a desired extent.

23. The apparatus of claim 19, wherein the flared conduit section is adapted to be laterally resilient allowing it to expand in its lateral direction and to mimic the function of the sinuses of Valsalva, while the conical shape of the flared conduit permits the formation of bulges during implantation to support eddy currents keeping the valve leaflets from impacting the wall of the conduit.

24. The apparatus of claim 19, wherein the flared end comprises a constant flare angle α ranging from about 5° to about 45°.

25. The apparatus of claim 19, wherein the flared end comprises a compound flare have a plurality of flared sections having different flare angles.

26. The apparatus of claim 19, wherein the flared end comprises a complex compound flare having sections having constant flare angle and section having varying flare angles.

27. The apparatus of claim 19, wherein the two section are made of the same or different materials.

28. The apparatus of claim 19, wherein the two section comprise material with orientable fibers.

29. The apparatus of claim 19, wherein the cylindrical section has fibers oriented to permit longitudinal elasticity and in the flared section fibers oriented to permit lateral elasticity.

30. The apparatus of claim 19, wherein the end section is cone-shaped

31. A method of manufacturing a prosthetic aortic conduit comprising the steps of:
providing a substantially uniform cylindrical section made of a first material suitable for use in heart surgery,
securing to an end of the cylindrical section, an outwardly flared section of a second material suitable for use in heart surgery so that the cylindrical section and the flared section align and smoothly transition from one to the other.

32. The method of claim 31, wherein the cylindrical conduit section has a longitudinal axis and optionally is resilient or has a resilient means allowing some expansion in its longitudinal direction,

33. The method of claim 31, wherein the flared section has a longitudinal axis and optionally is resilient or has a resilient means allowing the flared section to expand in its lateral direction

34. The method of claim 31, wherein the flared end comprises a constant flare angle α ranging from about 5° to about 45°.

35. The method of claim 31, wherein the flared end comprises a compound flare have a plurality of flared sections having different flare angles.

36. The method of claim 31, wherein the flared end comprises a complex compound flare having sections having constant flare angle and section having varying flare angles.

37. A method of implanting a prosthetic aortic conduit comprising the steps of:
providing a tubular conduit including:
a substantially straight and uniform section having a diameter d₁, and an outwardly flared section disposed at a distal end of the straight section having a length l and a diameter d₂ at the open flared end,
where the flared end is designed to be cut to closely mimic Valsalva sinuses of an aortic root of a patient to permit formation of bulges that expand radially outwardly and maintain a degree of flexibility in the longitudinal direction after implantation of the apparatus,
cutting the flared end to conform to the valve structure of a patient's aortic root before performing a aortic root replacement, and
performing the aortic root replacement so that bulges are formed in the implanted apparatus in conformity to the patient's aortic root.

38. The method of claim 37, wherein the flared end comprises a constant flare angle α ranging from about 5° to about 45°.

39. The method of claim 37, wherein the flared end comprises a compound flare have a plurality of flared sections having different flare angles.

40. The method of claim 37, wherein the flared end comprises a complex compound flare having sections having constant flare angle and section having varying flare angles.

41. The method of claim 37, wherein the length l is less than or equal to the diameter d₂.

42. The method of claim 37, wherein the diameter d₂ ranges from about 1.1×d₁ to about 1.5×d₁, regardless of the relationship between l and d₂.

43. The method of claim 37, wherein the diameter d₂ ranges from about 1.1×d₁ to about 1.4×d₁, regardless of the relationship between l and d₂.

44. The method of claim 37, wherein the d₂ ranges from about 1.1×d₁ to about 1.3×d₁, regardless of the relationship between l and d₂.

45. The method of claim 37, wherein the d₂ ranges from about 1.15×d₁ to about 1.3×d₁, regardless of the relationship between l and d₂.

46. The method of claim 37, wherein the length l is less than or substantially equal to the diameter d₂.

47. The method of claim 37, wherein the diameter d₁ is less than the diameter d₂ and the length l is less than or equal to the diameter d₂.

48. The method of claim 37, wherein the length l is greater than the diameter d₁, but shorter than the diameter d₂.
